(19) United States
(12) Patent Application Publication
(10) Pub. No.: US 2006/0190018 A1
(43) Pub. Date: Aug. 24, 2006

(54) Transoral endoscopic gastroesophageal flap valve restoration device having a guided tissue gripper

(75) Inventors: Steve G. Baker, Redmond, WA (US); Brett J. Carter, Monroe, WA (US); Clifton A. Alferness, Redmond, WA (US)

Correspondence Address: GRAYBEAL JACKSON HALEY LLP, Suite 350, 155-108th Avenue N.E., Bellevue, WA, 98004-5973, US

(73) Assignee: EsophyX, Inc.

(21) Appl. No.: 11/061,318

(22) Filed: Feb. 18, 2005

(51) Int. Cl. A61B 17/08

(52) U.S. Cl. 606/153

(57) ABSTRACT

A transoral gastroesophageal flap valve restoration device includes a tissue gripper which is guidable by one of a first and second member. The first and second members form a tissue shaper carried on a longitudinal member that shapes stomach tissue into the flap of a restored gastroesophageal flap valve.

### FIELD OF THE INVENTION

The present invention generally relates to devices for treating gastroesophageal reflux disease. The present invention more particularly relates to such devices which form a gastroesophageal flap valve flap and which includes a guide that guides a tissue gripper into engagement with stomach tissue.

### BACKGROUND

Gastroesophageal reflux disease (GERD) is a chronic condition caused by the failure of the anti-reflux barrier located at the gastroesophageal junction to keep the contents of the stomach from splashing into the esophagus. The splashing is known as gastroesophageal reflux. The stomach acid is designed to digest meat, and will digest esophageal tissue when persistently splashed into the esophagus.

A principal reason for regurgitation associated with GERD is the mechanical failure of a deteriorated gastroesophageal flap to close and seal against high pressure in the stomach. Due to reasons including lifestyle, a Grade I normal gastroesophageal flap may deteriorate into a malfunctioning Grade III or absent valve Grade IV gastroesophageal flap. With a deteriorated gastroesophageal flap, the stomach contents are more likely to be regurgitated into the esophagus, the mouth, and even the lungs. The regurgitation is referred to as “heartburn” because the most common symptom is a burning discomfort in the chest under the breastbone. Burning discomfort in the chest and regurgitation (burping up) of sour-tasting gastric juice into the mouth are classic symptoms of gastroesophageal reflux disease (GERD). When stomach acid is regurgitated into the esophagus, it is usually cleared quickly by esophageal contractions. Heartburn (backwashing of stomach acid and bile onto the esophagus) results when stomach acid is frequently regurgitated into the esophagus and the esophageal wall is inflamed.

Complications develop for some people who have GERD. Esophagitis (inflammation of the esophagus) with erosions and ulcerations (breaks in the lining of the esophagus) can occur from repeated and prolonged acid exposure. If these breaks are deep, bleeding or scarring of the esophagus with formation of a stricture (narrowing of the esophagus) can occur. If the esophagus narrows significantly, then food sticks in the esophagus and the symptom is known as dysphagia. GERD has been shown to be one of the most important risk factors for the development of esophageal adenocarcinoma. In a subset of people who have severe GERD, if acid exposure continues, the injured squamous lining is replaced by a precancerous lining (called Barrett's Esophagus) in which a cancerous esophageal adenocarcinoma can develop.

Other complications of GERD may not appear to be related to esophageal disease at all. Some people with GERD may develop recurrent pneumonia (lung infection), asthma (wheezing), or a chronic cough from acid backing up into the esophagus and all the way up through the upper esophageal sphincter into the lungs. In many instances, this occurs at night, while the person is in a supine position and sleeping. Occasionally, a person with severe GERD will be awakened from sleep with a choking sensation. Hoarseness can also occur due to acid reaching the vocal cords, causing a chronic inflammation or injury.

GERD never improves without intervention. Life style changes combined with both medical and surgical treatments exist for GERD. Medical therapies include antacids and proton pump inhibitors. However, the medical therapies only mask the reflux. Patients still get reflux and perhaps emphysema because of particles refluxed into the lungs. Barrett's esophagus results in about 10% of the GERD cases. The esophageal epithelium changes into tissue that tends to become cancerous from repeated acid washing despite the medication.

Several open laparotomy and laproscopic surgical procedures are available for treating GERD. One surgical approach is the Nissen fundoplication. The Nissen approach typically involves a 360° wrap of the fundus around the gastroesophageal junction. The procedure has a high incidence of postoperative complications. The Nissen approach creates a 360° moveable flap without a fixed portion. Hence, Nissen does not restore the normal movable flap. The patient cannot burp because the fundus was used to make the repair, and may frequently experience dysphagia. Another surgical approach to treating GERD is the Belsey Mark IV (Belsey) fundoplication. The Belsey procedure involves creating a valve by suturing a portion of the stomach to an anterior surface of the esophagus. It reduces some of the postoperative complications encountered with the Nissen fundoplication, but still does not restore the normal movable flap. None of these procedures fully restores the normal anatomical anatomy or produces a normally functioning gastroesophageal junction. Another surgical approach is the Hill repair. In the Hill repair, the gastroesophageal junction is anchored to the posterior abdominal areas, and a 180° valve is created by a system of sutures. The Hill procedure restores the moveable flap, the cardiac notch and the Angle of His. However, all of these surgical procedures are very invasive, regardless of whether done as a laproscopic or an open procedure.

New, less surgically invasive approaches to treating GERD involve transoral endoscopic procedures. One procedure contemplates a machine device with robotic arms that is inserted transorally into the stomach. While observing through an endoscope, an endoscopist guides the machine within the stomach to engage a portion of the fundus with a corkscrew-like device on one arm. The arm then pulls on the engaged portion to create a fold of tissue or radial plication at the gastroesophageal junction. Another arm of the machine pinches the excess tissue together and fastens the excess tissue with one pre-tied implant. This procedure does not restore normal anatomy. The fold created does not have anything in common with a valve. In fact, the direction of the radial fold prevents the fold or plication from acting as a flap of a valve.

Another transoral procedure contemplates making a fold of fundus tissue near the deteriorated gastroesophageal flap to recreate the lower esophageal sphincter (LES). The procedure requires placing multiple U-shaped tissue clips around the folded fundus to hold it in shape and in place.

This and the previously discussed procedure are both highly dependent on the skill, experience, aggressiveness, and courage of the endoscopist. In addition, these and other procedures may involve esophageal tissue in the repair. Esophageal tissue is fragile and weak. Involvement of esophageal tissue in the repair of a gastroesophageal flap valve poses unnecessary risks to the patient.

A new and improved device and method for restoration of a gastroesophageal flap valve is fully disclosed in U.S. Pat. No. 6,790,214, issued Sep. 14, 2004, for TRANSORAL ENDOSCOPIC GASTROESOPHAGEAL FLAP VALVE RESTORATION DEVICE, ASSEMBLY, SYSTEM AND METHOD, which patent is assigned to the assignee of this invention, and is incorporated herein by reference. That apparatus and method provides transoral endoscopic gastroesophageal flap valve restoration. A longitudinal member arranged for transoral placement into a stomach carries a tissue shaper that non-invasively grips and shapes stomach tissue. A tissue fixation device is then deployed to maintain the shaped stomach tissue in a shape approximating a gastroesophageal flap.

Whenever stomach tissue is to be transorally shaped into a gastroesophageal flap, as, for example, by the improved device last mentioned above, it is necessary to draw sufficient stomach tissue into the shaper to be assured that the resulting flap is long enough to cover the esophageal opening into the stomach. This requires gripping the stomach tissue at a point out and away from the esophageal opening. It also requires pulling the gripped tissue down (in an aboral direction) into the tissue shaper. Further, to form a complete gastroesophageal flap, this procedure must be repeated with rotation of the device and tissue shaping at a number of circumferentially spaced points.

To accomplish the above, it would be desirable if the gastroesophageal flap restoration device were able to guide the tissue gripper into engagement with the stomach tissue in a controlled manner to assure proper gripper positioning out and away from the esophageal opening into the stomach. To assist in this effort, it would be further desirable if the procedure could be viewable at all times. Still further, it would be helpful if the gripper were able to pull the gripped tissue down and into the tissue shaper while being later releasable from the tissue to permit the procedure to be repeated. The device preferably then redeploys and makes the gripper ready to once again be guided into engagement with the stomach tissue at its next location.

All of the forgoing functionality should be convenient to implement. This would enable the procedure to be completed in a timely manner and render a favorable outcome.

### SUMMARY

The invention provides a transoral gastroesophageal flap valve restoration device comprising a longitudinal member that is arranged for transoral placement into a stomach and a tissue shaper carried on the longitudinal member for placement in the stomach. The tissue shaper includes a pair of hingedly coupled first and second members that receive the stomach tissue there between and shapes the stomach tissue while the tissue is between the first and second members. The device further comprises a tissue gripper assembly including a gripper that grips and pulls the stomach tissue in between the first and second members. The gripper is guidable by one of the first and second members into engagement with the tissue.

The first member may comprise a chassis coupled to the longitudinal member, the second member may comprise a bail hingedly coupled to the chassis, and the gripper may be guided by the bail.

The gripper assembly may further comprise an elongated cable having a distal end. The gripper may be carried at the distal end of the cable. The cable may be confined by the bail as the gripper is guided into engagement with the tissue and releasable from the bail after gripping the tissue. The bail may have an opening permitting the cable and gripper to be drawn into the bail after the gripper disengages the gripped tissue. The gripper may be viewable through the bail while being guided into engagement with the tissue.

The gripper may comprise a helical coil. The helical coil may be formed by the cable or attached to the cable.

The present invention further provides a transoral gastroesophageal flap valve restoration device comprising a longitudinal member that is arranged for transoral placement into a stomach and a tissue shaper carried on the longitudinal member for placement in the stomach. The tissue shaper includes a chassis and a bail. The chassis and bail are hingedly coupled together to receive the stomach tissue there between and to shape the stomach tissue while the tissue is between the chassis and the bail. The device further comprises a tissue gripper assembly including a gripper that grips and pulls the stomach tissue in between the chassis and the bail. The bail includes a guide structure that guides the gripper into engagement with the tissue.

The gripper may be confined within the guide structure and viewable through the guide structure while being guided into engagement with the tissue.

The gripper assembly may further comprise an elongated cable having a distal end. The gripper may be carried at the distal end of the cable.

The guide structure may comprise a guide tube extending along the longitudinal dimension of the bail. The guide tube may have a slit that permits the gripper assembly cable to separate from the bail after the tissue is gripped by the gripper. The slit may have a circuitous configuration. The guide tube may include an opening that permits the gripper to be drawn into the guide tube after the cable has been separated from the bail and the gripper has released the gripped tissue.

The invention still further provides a transoral gastroesophageal flap valve restoration device comprising a longitudinal member that is arranged for transoral placement into a stomach and a tissue shaper carried on the longitudinal member for placement in the stomach. The tissue shaper includes a chassis and a bail. The bail is hingedly coupled to the chassis to receive the stomach tissue there between and to shape the stomach tissue. The device further comprises a tissue gripper assembly including a cable and a gripper that grips and pulls the stomach tissue in between the chassis and the bail. The bail includes a guide structure that acts upon the cable to guide the gripper into engagement with the stomach tissue and that disassociates from the cable after the gripper engages the tissue. The guide structure and the tissue gripper assembly are arranged to associate after the gripper disengages from the tissue.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like referenced numerals identify like elements, and wherein:

FIG. 1 is a front cross-sectional view of the esophageal-gastro-intestinal tract from a lower portion of the esophagus to the duodenum;

FIG. 2 is a front cross-sectional view of the esophageal-gastro-intestinal tract illustrating a Grade I normal appearance movable flap of the gastroesophageal flap valve (in dashed lines) and a Grade III reflux appearance gastroesophageal flap of the gastroesophageal flap valve (in solid lines);

FIG. 3 is a side view of a device according to an embodiment of the invention guiding a tissue gripper for engagement with stomach tissue to be shaped into a gastroesophageal flap;

FIG. 4 is a partial perspective view showing details of the tissue gripper guide of the device of the device of FIG. 3;

FIG. 5 is a side view similar to FIG. 3 showing the device after gripping the stomach tissue;

FIG. 6 is a side view similar to FIG. 3 showing the device open to receive the stomach tissue to be shaped and the tissue being pulled toward the device;

FIG. 7 is a partial perspective view similar to FIG. 4 showing details of the gripper being disassociated from the gripper guide as the stomach tissue to be shaped is pulled towards the device;

FIG. 8 is a side view similar to FIG. 3 showing the tissue to be shaped being pulled down into the device;

FIG. 9 is a view similar to FIG. 3 showing the gripped tissue being shaped by the device;

FIG. 10 is a side view similar to FIG. 3 showing the shaped tissue with the device open; and

FIG. 11 is a partial perspective view similar to FIG. 4 showing details of the gripper re-associating with the gripper guide in preparation for a repeat of the procedure shown in FIGS. 3-10.

### DETAILED DESCRIPTION

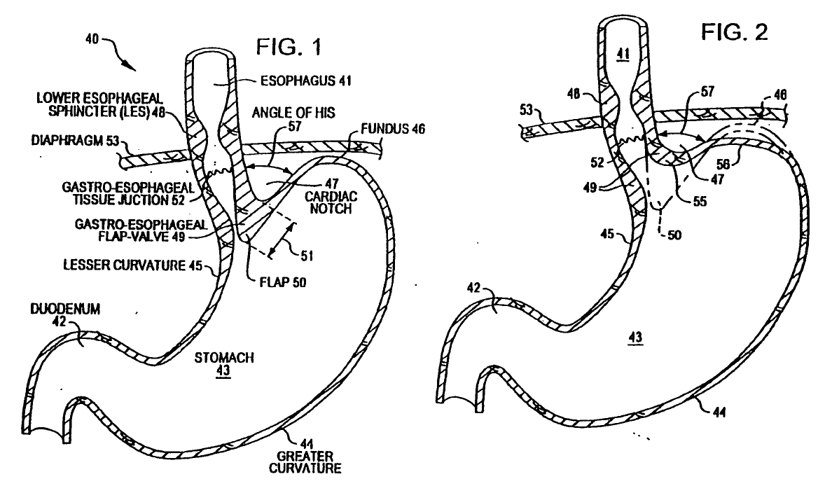

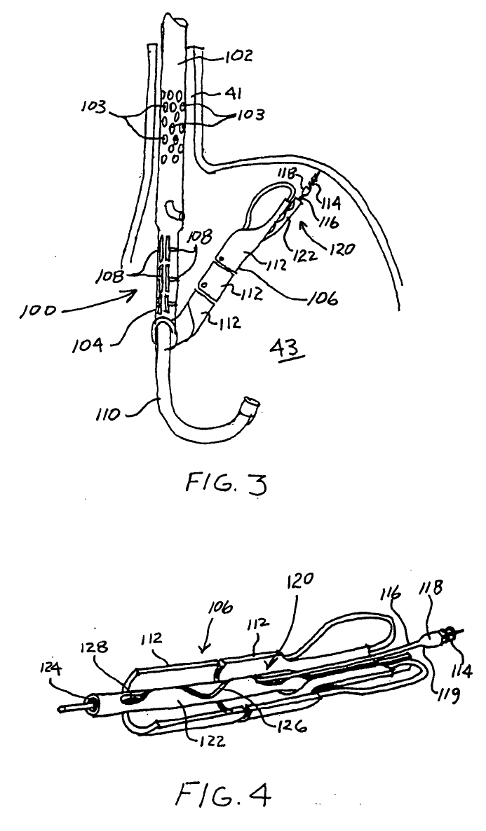

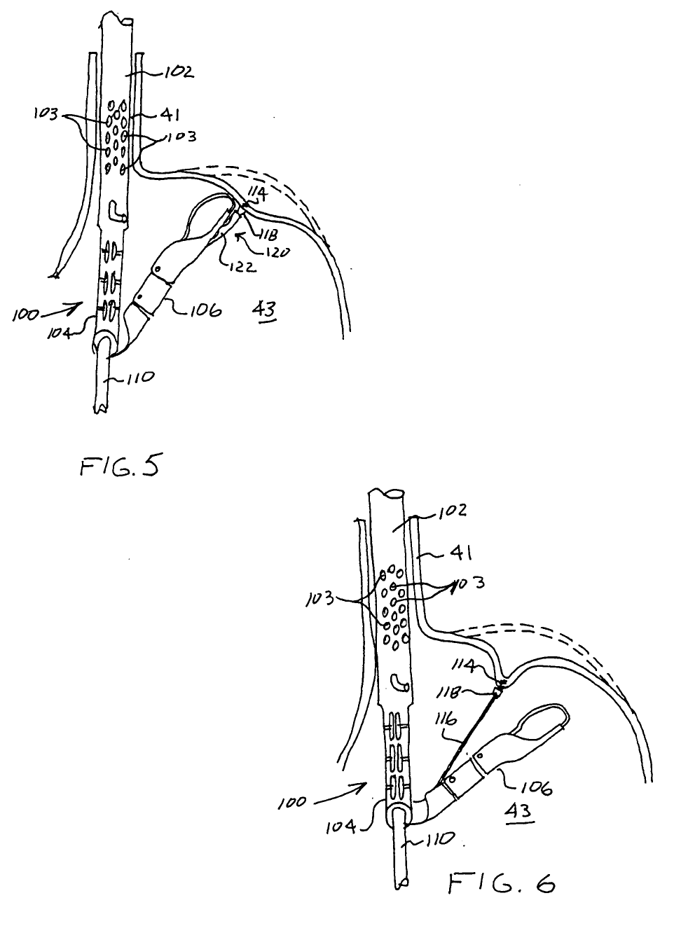

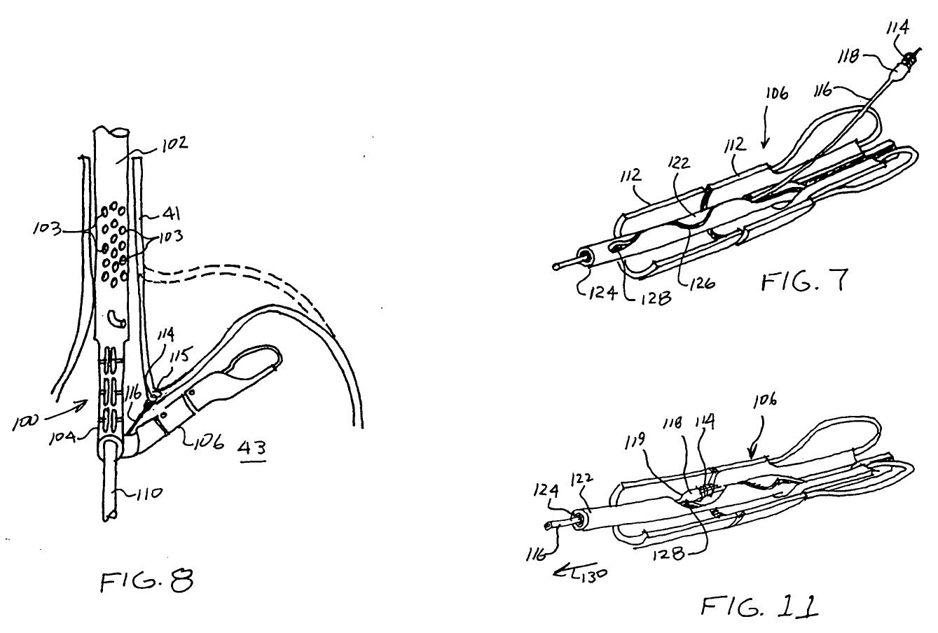

FIG. 1 is a front cross-sectional view of the esophageal-gastro-intestinal tract 40 from a lower portion of the esophagus 41 to the duodenum 42. The stomach 43 is characterized by the greater curvature 44 on the anatomical left side and the lesser curvature 45 on the anatomical right side. The tissue of the outer surfaces of those curvatures is referred to in the art as serosa tissue. As will be seen subsequently, the nature of the serosa tissue is used to advantage for its ability to bond to like serosa tissue. The fundus 46 of the greater curvature 44 forms the superior portion of the stomach 43, and traps gas and air bubbles for burping. The esophagus 41 enters the stomach 43 at an esophageal orifice below the superior portion of the fundus 46, forming a cardiac notch 47 and an acute angle with respect to the fundus 46 known as the Angle of His 57. The lower esophageal sphincter (LES) 48 is a discriminating sphincter able to distinguish between burping gas, liquids, and solids, and works in conjunction with the fundus 46 to burp. The gastroesophageal flap valve (GEFV) 49 includes a moveable portion and an opposing more stationary portion. The moveable portion of the GEFV 49 is an approximately 180°, semicircular, gastroesophageal flap 50 (alternatively referred to as a “normal moveable flap” or “moveable flap”) formed of tissue at the intersection between the esophagus 41 and the stomach 43. The opposing more stationary portion of the GEFV 49 comprises a portion of the lesser curvature 45 of the stomach 43 adjacent to its junction with the esophagus 41. The gastroesophageal flap 50 of the GEFV 49 principally comprises tissue adjacent to the fundus 46 portion of the stomach 43, is about 4 to 5 cm long (51) at its longest portion, and the length may taper at its anterior and posterior ends. The gastroesophageal flap 50 is partially held against the lesser curvature 45 portion of the stomach 43 by the pressure differential between the stomach 43 and the thorax, and partially by the resiliency and the anatomical structure of the GEFV 49, thus providing the valving function. The GEFV 49 is similar to a flutter valve, with the gastroesophageal flap 50 being flexible and closeable against the other more stationary side.

The esophageal tract is controlled by an upper esophageal sphincter (UES) in the neck near the mouth for swallowing, and by the LES 48 and the GEFV 49 at the stomach. The normal anti-reflux barrier is primarily formed by the LES 48 and the GEFV 49 acting in concert to allow food and liquid to enter the stomach, and to considerably resist reflux of stomach contents into the esophagus 41 past the gastroesophageal tissue junction 52. Tissue aboral of the gastroesophageal tissue junction 52 is generally considered part of the stomach because the tissue protected from stomach acid by its own protective mechanisms. Tissue oral of the gastroesophageal junction 52 is generally considered part of the esophagus and it is not protected from injury by prolonged exposure to stomach acid. At the gastroesophageal junction 52, the juncture of the stomach and esophageal tissues form a zigzag line, which is sometimes referred to as the “Z-line.” For the purposes of these specifications, including the claims, “stomach” means the tissue aboral of the gastroesophageal junction 52.

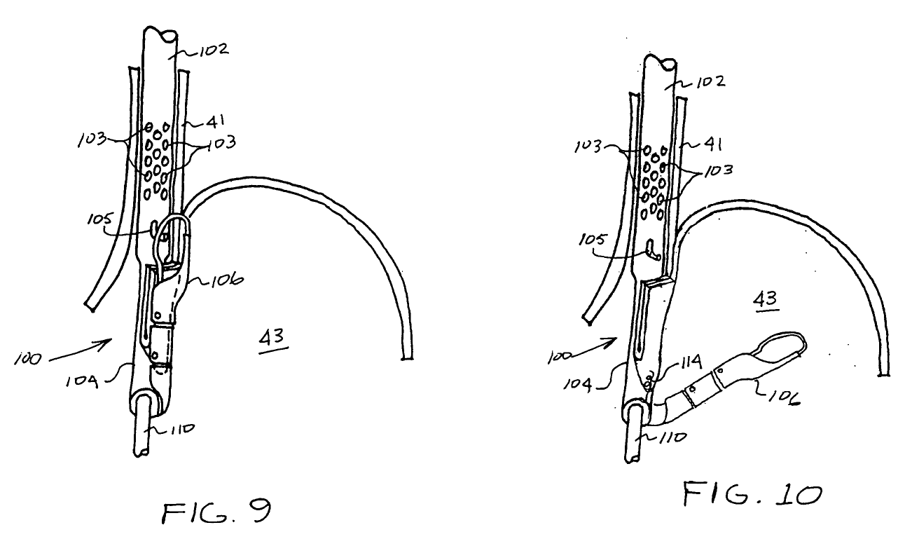

FIG. 2 is a front cross-sectional view of an esophageal-gastro-intestinal tract illustrating a Grade I normal appearance movable flap 50 of the GEFV 49 (shown in dashed lines) and a deteriorated Grade III gastroesophageal flap 55 of the GEFV 49 (shown in solid lines). As previously mentioned, a principal reason for regurgitation associated with GERD is the mechanical failure of the deteriorated (or reflux appearance) gastroesophageal flap 55 of the GEFV 49 to close and seal against the higher pressure in the stomach. Due to reasons including lifestyle, a Grade I normal gastroesophageal flap 50 of the GEFV 49 may deteriorate into a Grade III deteriorated gastroesophageal flap 55. The anatomical results of the deterioration include moving a portion of the esophagus 41 that includes the gastroesophageal junction 52 and LES 48 toward the mouth, straightening of the cardiac notch 47, and increasing the Angle of His 57. This effectively reshapes the anatomy aboral of the gastroesophageal junction 52 and forms a flattened fundus 56. The deteriorated gastroesophageal flap 55 illustrates a gastroesophageal flap valve 49 and cardiac notch 47 that have both significantly degraded. Dr. Hill and colleagues developed a grading system to describe the appearance of the GEFV and the likelihood that a patient will experience chronic acid reflux. L. D. Hill, et al., The gastroesophageal flap valve: in vitro and in vivo observations, Gastrointestinal Endoscopy 1996:44:541-547. Under Dr. Hill's grading system, the normal movable flap 50 of the GEFV 49 illustrates a Grade I flap valve that is the least likely to experience reflux. The deteriorated gastroesophageal flap 55 of the GEFV 49 illustrates a Grade III (almost Grade IV) flap valve. A Grade IV flap valve is the most likely to experience reflux. Grades II and III reflect intermediate grades of deterioration and, as in the case of III, a high likelihood of experiencing reflux. With the deteriorated GEFV represented by deteriorated gastroesophageal flap 55 and the fundus 46 moved inferior, the stomach contents are presented a funnel-like opening directing the contents into the esophagus 41 and the greatest likelihood of experiencing reflux. Disclosed subsequently is a device for restoring the normal gastroesophageal flap valve anatomy, which device is one embodiment of the present invention.

Referring now to FIG. 3, it shows a device 100 embodying the present invention. The device 100 includes a longitudinal member 102 for transoral placement into the stomach 43 through the esophagus 41. The device further includes a first member 104, hereinafter referred to as the chassis, and a second member 106, hereinafter referred to as the bail. The chassis 104 and bail 106 form a tissue shaper which, as described subsequently in accordance with the present invention, shapes tissue of the stomach 43 into the flap of a restored gastroesophageal flap valve. The chassis 104 and bail 106 are carried at the distal end of the longitudinal member 102 for placement in the stomach as illustrated.

The device 100 has a longitudinal passage to permit an endoscope 110 to be guided into the stomach. This permits the gastroesophageal flap valve restoration to be viewed at each stage of the procedure. [41] To facilitate shaping of the stomach tissue, the stomach tissue is drawn in between the chassis 104 and the bail 106. Further, to enable a flap of sufficient length to be formed to function as the flap of a gastroesophageal flap valve, the stomach tissue is pulled down so that the fold line is substantially juxtaposed to the opening of the esophagus 41 into the stomach 43. Hence, as will be seen, the stomach is first gripped at a point out and away from the esophagus 41 and the grip point is pulled to almost the hinged connection of the chassis 104 and bail 106. As described in copending application Ser. No. 11/001,666, filed Nov. 30, 2004, entitled FLEXIBLE TRANSORAL ENDOSCOPIC GASTROESOPHAGEAL FLAP VALVE RESTORATION DEVICE AND METHOD, which application is incorporated herein by reference, the device 100 is fed down the esophagus 41 with the bail 106 substantially in line with the chassis 104. To negotiate the bend of the throat, and as described in the aforementioned referenced application, the chassis 104 and bail 106 are rendered flexible. The chassis 104 is rendered flexible by the slots 108 and the bail 106 is rendered flexible by the hingedly coupled links 112. Further details concerning the flexibility of the chassis 104 and the bail 106 may be found in the aforementioned referenced application.

As further shown in FIG. 3, the device further includes a tissue gripper 114. The gripper 114, in this embodiment, comprises a helical coil. The gripper 114 is carried at the end of a cable 116 and may be attached to the end of the cable or be formed from the cable. In this embodiment, the helical coil is attached to the cable 116 and is preceded by a guide 118 whose function will be described subsequently.

The helical coil 114 is shown engaging the stomach tissue out and away from the opening of the esophagus 41 to the stomach 43. The helical coil 114 is being guided into position by a guide structure 120 carried on the bail 106. The guide structure 120 comprises a guide tube 122. When the device 100 is first introduced down the esophagus into the stomach, the helical coil 114 is caused to reside well within the guide tube 122 to preclude the helical coil from accidentally or inadvertently snagging esophageal or stomach tissue. The bail 106 and guide structure 120 are shown in greater detail in FIG. 4.

The guide tube 122 includes a lumen 124 which is sized to permit the helical coil 114 and the guide 118 to be longitudinally displaced through the lumen and just outside of the guide tube 122. The guide tube includes a longitudinal slit 126 having a circuitous configuration. The slit 126 as will be seen subsequently, permits the end of the cable to release or disassociate from the bail after the stomach tissue is gripped. The circuitous configuration of the slit 126 assures confinement of the cable 116 within the lumen 124 until release of the cable is desired. The proximal end of the slit 126 has an enlarged portion or opening 128. This opening permits the cable and helical coil to reenter the lumen when the device 100 is readied for a repeated stomach tissue shaping procedure. To that end, it may be noted that the guide 118 has a conical surface 119 that serves to guide the cable end back into the opening 128.

The bail 106 and guide tube 122 are preferably formed of a transparent or semi-transparent material. This permits the helical coil 114 to be viewed with the endoscope 110 as the helical coil 114 is advanced up through the guide tube into engagement with the stomach tissue.

When the helical coil 114 is to engage the stomach tissue as shown, for example, in FIG. 3, the helical coil 114 is first pushed into contact with the stomach tissue by pushing on the cable 116. This pushing on the cable 116 causes the cable to assume a sinusoidal or circuitous configuration within the guide tube 122. The wavelength of the cable at this time depends upon many factors and can be determined empirically. Preferably, the wavelength of the slit 126 is selected so as to be shorter than the wavelength of the cable 116 when it is pushed to cause the helical coil 114 to engage the stomach tissue.

When the stomach tissue is thus engaged by the helical coil 114 as shown in FIG. 3, the helical coil 114 may be rotated by rotating the cable 116. This will cause the helical coil to screw into the stomach tissue. Helical coils for this purpose are known. The gripped stomach tissue is rather thick, permitting the helical coil 114 to obtain a grip of the tissue without causing severe damage to the tissue. FIG. 5 shows the stomach tissue being gripped by the helical coil 114.

It may also be mentioned at this point that the longitudinal member 102 includes a plurality of orifices 103. These orifices 103 may be used to pull a vacuum to cause the longitudinal member 102 to grip the inner surface of the esophagus 41. This will serve to stabilize the esophagus 41 during the procedure. This vacuum gripping of the esophagus may be used to advantage if the patient suffers a hiatal hernia. Upon being thus gripped, the esophagus 41 may be moved downwardly toward the stomach to eliminate the hiatal hernia.

Once the stomach tissue is gripped as shown in FIG. 5, the bail 106 and chassis 104 are separated or open to permit the gripped tissue to be pulled between them. As the bail 106 is moved relative to the chassis 104, the cable 116 is caused to slip through the slit 126 of the guide tube 122. This is shown in FIG. 6.

FIG. 7 shows in greater detail the cable 116 being released from the bail 106 by sliding out of the guide tube 122 through the slit 126. This process continues until the cable 116 slides into the opening 128. A mechanism which may be employed in the device 100 to pivot the bail 106 relative to the chassis 104 for opening and closing these members is more fully described in copending application Ser. No. 11/001,666, filed Nov. 30, 2004, entitled FLEXIBLE TRANSORAL ENDOSCOPIC GASTROESOPHAGEAL FLAP VALVE RESTORATION DEVICE AND METHOD, which application is also incorporated herein by reference. The circuitous configuration of the slit 126 assures that the cable will only be released when desired by the opening of the bail 106 and chassis 104.

Referring now to FIG. 8, it may now be seen that the cable 116 and helical coil 114 have pulled the stomach tissue to well between the chassis 104 and bail 106. The gripping point 115 is substantially juxtaposed to the esophagus 41. The device 100 is now ready to close by rotating the bail 106 towards the chassis for shaping the stomach tissue.

FIG. 9 shows the stomach tissue being shaped by the chassis 104 and bail 106. It may be noted here that in FIGS. 9 and 10 a portion of the shaped stomach tissue has been cut away to better illustrate the operation of the device 100 in accordance with this embodiment. FIG. 10 provides an alternate view showing the shaped tissue with the device 100 opened.

With the stomach tissue confined between and shaped by the chassis 104 and bail 106, at least one fastener may be inserted through the formed tissue flap to maintain its shape. To this end, the device 100 further includes a channel 105 through which the fastener (not shown) may be fed and directed into the tissue. One such fastener which may be employed for this purpose is fully described in copending application Ser. No. 10/949,737, filed Sep. 23, 2004, entitled TISSUE FIXATION DEVICES AND ASSEMBLIES FOR DEPLOYING THE SAME. This copending application is also incorporated herein by reference.

Once the tissue has received the one or more fasteners, the helical coil 114 may be disengaged from the tissue. This may be done by rotating the helical coil in a reverse direction to cause it to unscrew from the tissue.

The cable 116 may now be drawn in a proximal direction as indicated by arrow 130 in FIG. 11. The conical sidewall of guide 118 associated with the helical coil 114 guides the helical coil 114 back within the lumen 124 to once again associate the cable 116 with the bail in preparation for a repeat of the procedure.

While particular embodiments of the present invention have been shown and described, modifications may be made, and it is therefore intended in the appended claims to cover all such changes and modifications which fall within the true spirit and scope of the invention.

What is claimed is:

1. A transoral gastroesophageal flap valve restoration device comprising:
a longitudinal member that is arranged for transoral placement into a stomach;
a tissue shaper carried on the longitudinal member for placement in the stomach, the tissue shaper comprising a pair of hingedly coupled first and second members that receive the stomach tissue there between and shapes the stomach tissue while the tissue is between the first and second members; and
a tissue gripper assembly including a gripper that grips and pulls the stomach tissue in between the first and second members, the gripper being guidable by one of the first and second members into engagement with the tissue.

2. The device of claim 1, wherein the first member comprises a chassis coupled to the longitudinal member, wherein the second member comprises a bail hingedly coupled to the chassis, and wherein the gripper is guided by the bail.

3. The device of claim 1, wherein the gripper assembly further comprises an elongated cable having a distal end, and wherein the gripper is carried at the distal end of the cable.

4. The device of claim 3, wherein the cable is confined by the bail as the gripper is guided into engagement with the tissue and releasable from the bail after gripping the tissue.

5. The device of claim 4, wherein the bail has an opening permitting the cable and gripper to be drawn into the bail after the gripper disengages the gripped tissue.

6. The device of claim 3, wherein the gripper comprises a helical coil.

7. The device of claim 6, wherein the helical coil is formed by the cable.

8. The device of claim 6, wherein the helical coil is attached to the cable.

9. The device of claim 3 wherein the gripper is attached to the cable.

10. The device of claim 3 wherein the gripper is formed from the cable.

11. The device of claim 3, wherein the gripper is confined within the bail and viewable through the bail while being guided into engagement with the tissue,

12. A transoral gastroesophageal flap valve restoration device comprising:
a longitudinal member that is arranged for transoral placement into a stomach;
a tissue shaper carried on the longitudinal member for placement in the stomach, the tissue shaper comprising a chassis and a bail, the chassis and bail being hingedly coupled together to receive the stomach tissue there between and to shape the stomach tissue while the tissue is between the chassis and the bail; and
a tissue gripper assembly including a gripper that grips and pulls the stomach tissue in between the chassis and the bail,
the bail including a guide structure that guides the gripper into engagement with the tissue.

13. The device of claim 12, wherein the gripper is confined within the guide structure and viewable through the guide structure while being guided into engagement with the tissue.

14. The device of claim 12, wherein the gripper assembly further comprises an elongated cable having a distal end, and wherein the gripper is carried at the distal end of the cable.

15. The device of claim 14, wherein the guide structure comprises a guide tube.

16. The device of claim 15 wherein the bail has a longitudinal dimension and wherein the guide tube extends along the longitudinal dimension of the bail.

17. The device of claim 16, wherein the guide tube has a slit that permits the gripper assembly cable to separate from the bail after the tissue is gripped by the gripper.

18. The device of claim 17, wherein the slit has a circuitous configuration.

19. The device of claim 16, wherein the guide tube includes an opening that permits the gripper to be drawn into the guide tube after the cable has been separated from the bail and the gripper has released the gripped tissue.

20. The device of claim 12, wherein the tissue gripper comprises a helical coil.

21. The device of claim 20, wherein the helical coil is formed from the cable.

22. The device of claim 20, wherein the helical coil is attached to the cable.

23. The device of claim 12, wherein the guide structure is arranged to permit the cable to separate therefrom after the tissue is gripped by the gripper.

24. The device of claim 23, wherein the guide structure is arranged to receive the cable and gripper therein after the gripped tissue is released by the gripper.

25. The device of claim 12, wherein the gripper is formed from the cable.

26. The device of claim 12, wherein the gripper is attached to the cable.

27. A transoral gastroesophageal flap valve restoration device comprising:
a longitudinal member that is arranged for transoral placement into a stomach;
a tissue shaper carried on the longitudinal member for placement in the stomach, the tissue shaper comprising a chassis and a bail, the bail hingedly coupled to the chassis to receive the stomach tissue there between and to shape the stomach tissue; and
a tissue gripper assembly including a cable and a gripper that grips and pulls the stomach tissue in between the chassis and the bail,
the bail including a guide structure that acts upon the cable to guide the gripper into engagement with the stomach tissue and that disassociates from the cable after the gripper engages the tissue.

28. The device of claim 27, wherein the gripper is confined within the guide structure and viewable through the guide structure while being guided into engagement with the tissue.

29. The device of claim 27, wherein the gripper comprises a helical coil.

30. The device of claim 27 wherein the gripper is attached to the cable.

31. The device of claim 27 wherein the gripper is formed from the cable.

32. The device of claim 27, wherein the guide structure and the tissue gripper assembly are arranged to associate after the gripper disengages from the tissue.
